(19) United States
(12) Patent Application Publication
(10) Pub. No.: US 2009/0258037 A1
(43) Pub. Date: Oct. 15, 2009

(54) VACCINE DEVELOPMENT STRATEGY USING MICROGRAVITY CONDITIONS

(75) Inventors: Timothy G. Hammond, New Orleans, LA (US); Patricia L. Allen, Tullahoma, TN (US)

Correspondence Address: KLARQUIST SPARKMAN, LLP, 121 SW SALMON STREET, SUITE 1600, PORTLAND, OR, 97204, US

(73) Assignee: The Government of the United States of America as represented by the Department of Veterans Affairs

(21) Appl. No.: 12/411,313

(22) Filed: Mar. 25, 2009

Related U.S. Application Data
(60) Provisional application No. 61/039,731, filed on Mar. 26, 2008.

(51) Int. Cl. A61K 39/02; C12Q 1/68; C40B 30/06; C12N 1/20

(52) U.S. Cl. 424/234.1; 435/6; 506/10; 435/252.8

(57) ABSTRACT

Methods are provided herein for producing a vaccine, comprising culturing bacteria in microgravity. In some examples, the method includes culturing bacteria in microgravity, evaluating RNA expression, detecting an RNA that is over- or underexpressed during culture in microgravity, deleting the over- or underexpressed RNA in bacteria, and killing or attenuating the bacteria to produce a vaccine. In other examples, the method comprises culturing bacteria in microgravity, evaluating RNA expression, detecting a RNA that is over- or underexpressed during culture in microgravity, selecting bacteria that over- or underexpress the RNA, culturing the selected bacteria, and killing the bacteria to produce a vaccine. Vaccine compositions produced by the disclosed methods are also contemplated.

### CROSS REFERENCE TO RELATED APPLICATION

This application claims the benefit of U.S. Provisional Application No. 61/039,731, filed Mar. 26, 2008, which is incorporated by reference herein in its entirety.

### FIELD

This disclosure relates to methods for producing a vaccine using microgravity conditions, which include culturing bacteria in microgravity conditions, evaluating RNA expression, and detecting an RNA that is over- or underexpressed during culture in microgravity.

### BACKGROUND

Infectious disease is a leading cause of death worldwide, having a major impact on global economy and security. In the United States alone, the total cost of managing infectious disease is in excess of $120 billion per annum, due to direct expenditures associated with delivery of care and medical interventions, as well as loss of productivity from the workforce. Moreover, increasing antibiotic resistance, combined with intentional misuse of microbial pathogens as weapons of bioterrorism underscores the need for more effective prevention and treatment of infectious agents, including development of vaccines.

Salmonella enterica (subtype Typhi) is a common bacteria found world-wide. This organism is the cause of typhoid fever, which plagued the United States in the nineteenth and early twentieth centuries. This frequently fatal disease is contracted through contaminated food and water, but occasional asymptomatic carriers like the infamous “Typhoid Mary” also spread disease. Improved hygiene and surveillance have now virtually eliminated the threat of typhoid fever in the United States. However, milder Salmonella illnesses are one of the largest contributors to food-borne disease in the United States, and because of the large numbers of persons affected, mortality from this syndrome is significant. Salmonella infection is still one of the most common forms of food poisoning in the United States, and the loss of productivity from this syndrome is estimated at close to $100 billion annually. Notably, the first reported incident of bioterrorism in the United States involving food occurred when a group purposefully contaminated salad bars with Salmonella, resulting in more than 700 illnesses.

Closely related strains of the same Salmonella species that caused typhoid fever now produce diarrheal disease with less severe symptoms and outcomes, but orders of magnitude greater incidence. These strains became endemic in commercial chicken populations, and most outbreaks of Salmonella gastroenteritis are associated with undercooked poultry or eggs. In addition, several recent large outbreaks have been traced back to rather unusual sources involving unpasteurized orange juice, salad tomatoes, spinach, and/or cantaloupes. The association of these products with Salmonella disease is of growing concern because they are usually consumed without cooking. Worldwide, Salmonella diarrhea remains one of the top three causes of infant mortality, and a vaccine has the potential to make dramatic improvements in the third world incidence of this disease. Thus, there is a need to develop vaccines against Salmonella and other infectious agents.

### SUMMARY

Methods are provided herein for producing a vaccine by culturing bacteria in microgravity (including simulated microgravity) conditions. In some embodiments, the bacteria cultured in microgravity are Salmonella bacteria, such as S. enterica, (for example, S. enterica serovar Enteritidis).

In the methods disclosed herein, bacteria is cultured in microgravity conditions and the expression of RNAs (such as mRNA or small RNA (sRNA)) is evaluated, and one or more RNA is detected that exhibits a change in expression level during culture in microgravity as compared to during culture in normal gravity. In some examples, a nucleic acid encoding an RNA that is overexpressed or underexpressed during culture in microgravity is deleted in a bacterial population and the bacteria is attenuated or killed, producing a vaccine. In other examples, bacteria that overexpress or underexpress an RNA during culture in microgravity are selected and cultured, producing a bacterial strain, which is then killed for use as a vaccine. In particular examples, the change in RNA expression increases bacterial virulence.

The disclosed methods include culturing bacteria in microgravity conditions. In some examples, the effects of microgravity conditions are reproduced by the conditions of cell culture, such as by balancing gravity with equal and opposite forces to create simulated microgravity. In other examples, microgravity is produced by spaceflight, such as on a space shuttle or the International Space Station.

In some examples, the disclosed methods include the culture of bacteria from the family Enterobacteriaceae. In particular examples, the disclosed methods utilize the bacterium Salmonella enterica (for example, S. enterica serovar Enteritidis).

The methods disclosed herein include detecting changes in expression of RNA in bacteria cultured in microgravity as compared to in bacteria cultured in normal gravity (for example, using microarray analysis). In some examples, the RNA is a sRNA, including, but not limited to, IstR, InvR, DsrA, SsrS, MicA, MicC, MicF, SroB, RybB, SraH, RprA, SgrS, GcvB, or any combination thereof. In additional examples, the RNA is an mRNA, including, but not limited to, HilA, HilD, RhuM, PipA, or any combination thereof.

Additional embodiments include vaccines that are produced by the claimed methods.

The foregoing and other features and advantages of the invention will become more apparent from the following detailed description of a several embodiments which proceeds with reference to the accompanying figures.

### BRIEF DESCRIPTION OF THE FIGURES

FIG. 1A is a schematic Map of the 4.8-Mb circular S. typhimurium genome with the locations of the genes belonging to the space flight transcriptional stimulon indicated as black hash marks.

FIG. 1B is plot showing decreased time to death in mice infected with flight S. typhimurium as compared with identical ground controls. Female BALB/c mice per-orally infected with 107 bacteria from either space flight or ground cultures were monitored every 6-12 h over a 30-day period, and the percent survival of the mice in each group is graphed versus the number of days.

FIG. 1C is a bar graph showing increased percent mortality of mice infected with space flight cultures across a range of infection dosages. Groups of mice were infected with increasing dosages of bacteria from space flight and ground cultures and monitored for survival over 30 days. The percent mortality of each dosage group is graphed versus the dosage amount.

FIG. 1D is a bar graph showing decreased LD₅₀ value for space flight bacteria in a murine infection model.

FIG. 1E is digital image of a scanning electron micrograph (SEM) of space flight and ground S. typhimurium bacteria showing the formation of an extracellular matrix and associated cellular aggregation of space flight cells. (Magnification: ×3,500.)

### DETAILED DESCRIPTION

### I. Abbreviations and Terms

Unless otherwise explained, all technical and scientific terms used herein have the same meaning as commonly understood by one of ordinary skill in the art to which this disclosure belongs. The singular terms “a,” “an,” and “the” include plural referents unless the context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.” All publications, patent applications, patents, and other references mentioned herein are incorporated by reference in their entirety for all purposes. In case of conflict, the present specification, including explanations of terms, will control. In addition, the materials, methods, and examples are illustrative only and not intended to be limiting.

Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

CFU: colony forming unit

FPA: fluid processing apparatus

GFP: green fluorescent protein

LSMMG: low-shear modeled microgravity

LD₅₀: lethal dose 50%

mRNA: messenger RNA

RWV: rotating wall vessel

sRNA: small RNA

Bacteria: Prokaryotic, single-cellular microorganisms. In some examples, bacteria include members of the family Enterobacteriaceae, such as Enterobacter, Escherichia, Klebsiella, Proteus, Salmonella, Shigella, and Yersinia. Bacteria also include other medically important bacteria, such as Staphylococcus (for example, Staphylococcus aureus, such as methicillin resistant S. aureus (MRSA)), Streptococcus sp., Enterococcus sp., and Pseudomonas sp.

Culture of bacteria: A population of bacteria, such as pathogenic or potentially pathogenic bacteria, that is grown in a defined set of conditions (such as gravitational field, temperature, culture medium, and/or time of culture). In some examples, a culture of bacteria includes a substantially pure culture (for example, Salmonella sp., Salmonella enterica, or S. enterica serovar Enteritidis). In additional examples, a culture of bacteria includes a mixed culture, such as co-culture of two or more bacterial species (for example E. coli, S. enterica, and/or S. gallinarum), two or more bacterial strains or serovars of the same species (such as S. enterica serovar Typhi, S. enterica serovar Typhimurium, and/or S. enterica serovar Enteritidis), or a combination thereof. In further examples, a culture of bacteria includes co-culture of bacteria with one or more other organisms, such that a characteristic of the bacteria can be evaluated (for example, co-culture of S. enterica with C. elegans to facilitate determining virulence of the cultured S. enterica).

Enterobacteriaceae: A family of rod-shaped, Gram-negative bacteria, which includes many members of the gut flora of humans and other animals, as well as numerous pathogenic bacteria. The Enterobacteriaceae include, but are not limited to, Enterobacter, Escherichia, Klebsiella, Proteus, Salmonella, Shigella, and Yersinia.

Immunogenic Composition: Terms used herein to mean a composition useful for stimulating or eliciting a specific immune response (or immunogenic response) in a vertebrate. In some embodiments, the immunogenic response is protective or provides protective immunity, in that it enables the vertebrate animal to better resist infection or disease progression that results from infection with the organism against which the immunogenic composition is directed. In other embodiments, be for the treatment of an existing condition.

In an embodiment, the immunogenic composition can be directed to a pathogenic or potentially pathogenic bacteria. For example, it is believed that an immunogenic response can arise from the generation of an antibody specific to one or more of the epitopes provided in the immunogenic composition. The response can include a T-helper or cytotoxic cell-based response to one or more of the epitopes provided in the immunogenic composition. All of these responses may originate from naïve or memory cells. A response can also include production of cytokines. One specific example of a type of immunogenic composition is a vaccine. An immunogenic composition is also referred to as an immune-stimulating composition.

Microgravity: A state in which there is very little net gravitational force, for example, gravity less than about 0.01×g. Microgravity conditions exist in space, for example, aboard the Space Shuttle, the International Space Station, a satellite, or a rocket while in flight outside the Earth's atmosphere. Simulated microgravity is microgravity which is simulated by a set of Earth-based conditions that mimic microgravity, such as by balancing gravity with equal and opposite forces (for example, shear force, centipedal force, Coriolus forces, buoyancy, and/or magnetic field). In one example, simulated microgravity may be generated by use of a clinostat, such as a rotating wall vessel (RWV). The term “microgravity conditions” and “microgravity” are used synonymously herein. Normal gravity is the gravity normally experienced on Earth, such as on the surface of the Earth and/or in its atmosphere (for example, in aircraft in the atmosphere of the Earth). Gravity is measured in terms of acceleration due to gravity, denoted by g. The strength (or apparent strength) of Earth's gravity varies with latitude, altitude, local topography, and geology. In some examples, normal gravity (such as 1×g) is about 9-10 m/s², for example, about 9.7-9.9 m/s². In particular preferred embodiments, normal gravity is that experienced on the surface of the Earth under normal gravity at that location on the Earth.

Overexpress: Increase in amount of a nucleic acid (such as a small RNA or an mRNA) or a polypeptide as compared to a control sample. The increase can be about 20%, about 30%, about 40%, about 50%, about 60%, about 70%, about 80%, about 90%, about 100%, about 200%, about 300%, about 400%, about 500% or even grated than about 500%. In some examples, an overexpressed RNA is one in which the amount of an RNA present in bacteria cultured in microgravity or simulated microgravity is increased as compared with the amount of the same RNA present in bacteria cultured in normal gravity.

Pharmaceutically acceptable carriers: The pharmaceutically acceptable carriers useful in the methods disclosed herein are conventional. Remington's Pharmaceutical Sciences, by E. W. Martin, Mack Publishing Co., Easton, Pa., 15th Edition (1975), describes compositions and formulations suitable for pharmaceutical delivery of the compositions herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol, or the like as a vehicle. For solid compositions (for example, powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain non-toxic auxiliary substances, such as wetting or emulsifying agents, preservatives, salts, amino acids, and pH buffering agents and the like, for example sodium or potassium chloride or phosphate, TWEEN®, sodium acetate or sorbitan monolaurate.

RNA (ribonucleic acid): RNA is a long chain polymer consisting of nucleic acids joined by 3′-5′ phosphodiester bonds. The repeating units in RNA polymers are four different nucleotides, each of which comprises one of the four bases, adenine, guanine, cytosine, and uracil bound to a ribose sugar to which a phosphate group is attached. In general, DNA is transcribed to RNA by an RNA polymerase. RNA transcribed from a particular gene contains both introns and exons of the corresponding gene; this RNA is also referred to as pre-mRNA. RNA splicing subsequently removes the intron sequences and generates a messenger RNA (mRNA) molecule, which can be translated into a polypeptide. Triplets of nucleotides (referred to as codons) in an mRNA molecule code for each amino acid in a polypeptide, or for a stop signal. Additional types of RNA molecules include transfer RNA (tRNA), which are involved in translation of RNA into protein, ribosomal RNA (rRNA), which are components of the ribosome, small nuclear RNA (snRNA), which are involved in RNA splicing, ribozymes, which are catalytic RNAs, and small non-coding RNA (sRNA).

Rotating Wall Vessel (RWV): A rotating bioreactor for cell culture which is optimized to produce laminar flow and minimize mechanical stress on cells in culture. In the RWV system, the force of gravity is counterbalanced by mechanical forces, thereby simulating microgravity conditions. When the axis of the RWV bioreactor's rotation is perpendicular to the gravitational vector, simulated microgravity is achieved. If the axis of rotation is parallel to the gravitational vector, a condition of 1×g (normal gravity) is achieved in the RWV.

Salmonella: Bacteria which are members of the family Enterobacteriaceae and the genus Salmonella. In one example, this includes Salmonella enterica serovar Typhi (also called Salmonella Typhi), the causative agent of typhoid fever. In another example, Salmonella includes Salmonella enterica serovar Enteritidis (also called Salmonella Enteritidis), which has become the single most common cause of food poisoning in the United States. In other examples, Salmonella includes additional Salmonella species, such as S. gallinarum, S. dublin, S. abortus-equi, S. abortus-ovi, S. choleraesuis, and S. arizonae.

Small RNA (sRNA): Small non-coding RNAs, typically about 50-500 nucleotides in length, which do not commonly contain an expressed open reading frame. It is estimated that enterobacterial genomes contain 200-300 sRNA genes (Vogel and Papenfort, Curr. Opin. Microbiol. 9:605-611, 2006). Particular examples of sRNAs include, but are not limited to, InvR, IstR, DsrA, SsrS, MicA, MicC, MicF, SroB, RybB, SraH, RprA, SgrS, GcvB, CsrB, and CsrC.

Many sRNAs function by direct base-pairing with a target mRNA and affecting mRNA stability or ability to be translated. Some sRNAs act by inhibiting mRNA translation, such as MicA, OxyS, and SgrS. Other sRNAs positively regulate mRNA translation, for example DsrA and RprA. In other cases, some sRNAs modify protein activity; for example, CsrB and CsrC sRNAs bind the translational regulatory protein CsrA and titrate it away from its mRNA target sites. See, for example Gerhart et al., Noncoding RNAs Encoded by Bacterial Chromosomes, in Noncoding RNAs: Molecular Biology and Molecular Medicine (eds. Barciszewski and Erdmann), Eurekah, 2003.

sRNAs are expressed in many of the Enterobacteriaceae, such as Escherichia coli, Salmonella enterica, Shigella flexneri, Yersinia pestis, Erwinia carotovora, Klebsiella pneumoniae, Serratia marcescens, Photorhabdus luminescens, and Citrobacter rodentium.

Spaceflight: Travel outside of the Earth's atmosphere, for example on the Space Shuttle, the International Space Station, a satellite, a rocket, or other space vehicle, such that microgravity conditions exist. Spaceflight includes travel in Earth orbit, as well as travel through space, such as between planets.

Subject: Living multi-cellular organisms, a category that includes human and non-human animals, such as laboratory or veterinary subjects (for example, primates, cows, rodents (such as mice and rats), and chickens). Subjects further include invertebrate organisms (such as Caenorhabditis elegans).

Underexpress: Decrease in amount of a nucleic acid (such as a small RNA or an mRNA) or a polypeptide as compared to a control sample. The decrease can be about 20%, about 30%, about 40%, about 50%, about 60%, about 70%, about 80%, about 90%, about 100%, about 200%, about 300%, about 400%, about 500% or even grated than about 500%. In some examples, an underexpressed RNA is one in which the amount of an RNA present in bacteria cultured in microgravity or simulated microgravity is decreased as compared with the amount of the same RNA present in bacteria cultured in normal gravity.

Vaccine: A preparation of immunogenic material capable of stimulating an immune response, administered for the prevention, amelioration, or treatment of infectious or other types of disease. The immunogenic material may include attenuated or killed microorganisms (such as bacteria or viruses), or antigenic proteins, peptides or DNA derived from them. An attenuated vaccine is a virulent organism that has been modified to produce a less virulent form, but nevertheless retains the ability to elicit antibodies and cell-mediated immunity against the virulent form. A killed vaccine is a previously virulent microorganism that has been killed with chemicals or heat, but elicits antibodies against the virulent microorganism. Vaccines may elicit both prophylactic (preventative) and therapeutic responses. Methods of administration vary according to the vaccine, but may include inoculation, ingestion, inhalation or other forms of administration. Vaccines may be administered with an adjuvant to boost the immune response.

Virulence: The degree or ability of a pathogenic organism (such as bacteria or virus) to cause disease. Methods for assessing virulence include determining microbial resistance to acid stress, resistance to killing following uptake by macrophages, and killing of host organisms (such as mice or C. elegans). Virulence may also be assessed in cell-based assays, such as bacterial invasion of or adhesion to cells in monolayer or suspension culture.

### II. Overview of Several Embodiments

A. Methods for Identification of an Immunogenic Composition

Methods are provided herein for identifying and producing an immunogenic composition. Such methods include culturing bacteria in microgravity, for example the microgravity experienced in spaceflight. In some embodiments, the bacteria are Salmonella bacteria, such as S. enterica, (for example, S. enterica serovar Enteritidis).

In the methods disclosed herein, bacteria are cultured in microgravity conditions, for example during spaceflight. The expression of RNA from bacteria cultured in microgravity conditions is evaluated, one or more RNA is detected that is differentially expressed during culture in microgravity as compared to during culture in normal gravity, and the nucleic acid encoding the differentially expressed RNA is deleted in bacteria, producing a deleted bacterial strain. The deleted bacterial strain may be attenuated or killed, producing a vaccine. In some examples, the changes in RNA expression increase bacterial virulence, and deletion of the identified RNA yields an attenuated strain suitable for use as a vaccine, either directly or as an adjuvant.

Also disclosed herein are methods for producing an immunogenic composition in which bacteria is cultured in microgravity, the expression of RNA from bacteria cultured in microgravity conditions is evaluated, and one or more RNA is detected that is differentially expressed during culture in microgravity as compared to during culture in normal gravity is detected. A bacteria is selected that differentially expresses one or more RNA during culture in microgravity conditions and the bacteria is cultured, and killed, producing a killed vaccine.

In some examples, the differential expression of RNA during culture in microgravity includes overexpression of one or more RNAs as compared to bacteria cultured in normal gravity. In additional examples, the differential expression of RNA during culture in microgravity conditions includes underexpression of one or more RNAs as compared to bacteria cultured in normal gravity. In further examples, the differential expression of RNA during culture in microgravity may include overexpression of one or more RNAs and underexpression of one or more RNAs as compared to bacteria cultured in normal gravity.

The disclosed methods include culturing bacteria in microgravity. In some examples, microgravity is simulated microgravity produced by the conditions of cell culture, such as in a clinostat, for example a RWV. In other examples, microgravity is produced by spaceflight, such as on a space shuttle or the International Space Station.

In some examples, bacteria cultured in microgravity conditions and normal gravity can be bacteria taken from a culture of a single bacterial isolate, such as an isolate substantially or completely free of any other bacteria, such as isolates obtained from American Type Culture Collection or from standard laboratory strains. Thus, substantially similar bacteria can be cultured both in microgravity and standard gravity environment and the difference in expression of RNA between the bacteria grown in the two environments can be evaluated. The disclosed methods include the culture of bacteria (such as a substantially pure culture or monoculture of the target bacteria of interest, or a co-culture of the target bacteria with one or more other organisms, such as an organism that can be used to evaluate bacterial virulence (for example, C. elegans)). In some examples, the bacteria are pathogenic or potentially pathogenic bacteria. In some examples, the bacteria are from the family Enterobacteriaceae (such as Salmonella, Enterobacter, Escherichia, Klebsiella, Proteus, Shigella, and Yersinia). In particular examples, the disclosed methods utilize the bacterium Salmonella enterica (for example, S. enterica serovar Enteritidis). The methods disclosed herein may also be applied to fungal pathogens, including Candida sp., Blastomyces dermatitidis, Coccidioides immitis, Histoplasma capsulatum, Paracoccidioides brasiliensis and Penicillium marneffei.

The methods disclosed herein include detecting the over- or underexpression of sRNA in bacteria cultured in microgravity as compared to in bacteria of the same species, or species and serovar, cultured in normal gravity. In some examples, the sRNA detected includes IstR, InvR, DsrA, SsrS, MicA, MicC, MicF, SroB, RybB, SraH, RprA, SgrS, GcvB, or any combination thereof. In some examples, overexpression or underexpression of a sRNA increases virulence of the bacteria. In additional examples, the methods include deleting a nucleic acid encoding a sRNA that is over- or underexpressed in culture in microgravity, producing a deleted bacterial strain. These deleted bacterial strains include S. enterica with a deletion of the nucleic acid encoding IstR, InvR, DsrA, SsrS, MicA, MicC, MicF, SroB, RybB, SraH, RprA, SgrS, GcvB, or any combination thereof. Deleted bacterial strains may yield attenuated strains that are useful as a vaccine, or deleted bacterial strains may be killed or attenuated to produce a vaccine.

The methods disclosed herein include detecting the over- or underexpression of mRNA in bacteria cultured in microgravity as compared to in bacteria of the same species, or species and serovar, cultured in normal gravity. In some examples, the mRNA detected includes HilA, HilD, RhuM, PipA, or any combination thereof. In some examples, over- or underexpression of a mRNA increases virulence of the bacteria. In additional examples, the methods include deleting a nucleic acid encoding a mRNA that is overexpressed or underexpressed in culture in microgravity, producing a deleted bacterial strain. These deleted bacterial strains include S. enterica with a deletion of the nucleic acid encoding HilA, HilD, RhuM, PipA, or any combination thereof. Deleted bacterial strains may yield attenuated strains that are useful as a vaccine, or deleted bacterial strains may be killed or attenuated to produce a vaccine.

Additional embodiments include vaccines that are produced by the methods disclosed herein.

B. Bacteria and Culture Conditions

Bacteria which may be used in the disclosed methods include pathogenic or potentially pathogenic bacteria. In some examples, bacteria include members of the family Enterobacteriaceae. This is a family of rod-shaped, Gram-negative bacteria, which includes many members of the gut flora of humans and other animals, as well as numerous pathogenic bacteria. The Enterobacteriaceae include, but are not limited to, Enterobacter (for example, Enterobacter cloacae), Escherichia (for example, Escherichia coli), Klebsiella (for example, Klebsiella pneumoniae), Proteus (for example, Proteus mirabilis or P. vulgaris), Salmonella (for example, Salmonella enterica (such as serovars Typhimurium or Enteritidis), or S. bongori), Shigella (for example, Shigella flexneri, S. dysenteriae, or S. sonnei), and Yersinia (for example, Yersinia pestis).

Additional bacteria that may be used in the described methods include Staphylococcus (for example, Staphylococcus aureus, such as methicillin resistant S. aureus (MRSA)), Streptococcus (for example, Streptococcus pneumoniae), Enterococcus (for example Enteroccocus faecalis), and Pseudomonas sp.

In some examples, bacteria used in the disclosed methods include members of the genus Salmonella. This genus includes Salmonella enterica serovar Typhi (also called Salmonella Typhi or abbreviated to S. Typhi), which is the causative agent of typhoid fever. Although typhoid fever is not widespread in the United States, it is very common in under-developed countries, and causes a serious, often fatal disease. The symptoms of typhoid fever include nausea, vomiting, fever and death. S. Typhi can only infect humans, and no other host has been identified. The main source of S. Typhi infection is from swallowing contaminated water; food may also be contaminated with S. Typhi, if it is washed or irrigated with contaminated water. Salmonella enterica serovar Typhimurium (also called Salmonella Typhimurium or abbreviated to S. Typhimurium) is also a member of the genus Salmonella and causes disease in rodents, especially mice. Until recently, this serovar was the most common cause of food poisoning by Salmonella species was due to S. Typhimurium. This bacterium is capable of infecting mice and causes a typhoid-like disease in mice. In humans S. Typhimurium does not cause as severe disease as S. Typhi, and is not normally fatal. The disease is characterized by diarrhea, abdominal cramps, vomiting and nausea, and generally lasts up to 7 days. In immunocompromized people, that is the elderly, young, or people with depressed immune systems, S. Typhimurium infections are often fatal if they are not treated with antibiotics. The third member of the genus Salmonella is Salmonella enterica serovar Enteritidis (also called Salmonella Enteritidis or abbreviated to S. Enteritidis). Recently, S. Enteritidis has become the single most common cause of food poisoning in the United States, causing a disease almost identical to the very closely related S. Typhimurium. This serovar is capable of infecting mice and C. elegans, in addition to humans. S. Enteritidis is particularly adept at infecting chicken flocks without causing visible disease, and spreading from hen to hen rapidly. This bacterium has also been responsible for recent outbreaks of disease associated with contaminated orange juice, tomatoes, and spinach. In some examples, the bacteria utilized in the disclosed methods include S. enterica wild type strain SL1344.

The methods disclosed herein include culturing bacteria (such as a substantially pure monoculture of the target bacteria of interest, or a co-culture of the target bacteria with one or more other organisms, such as an organism that can be used to evaluate bacterial virulence (for example, C. elegans)) in microgravity conditions. Most modern cell culture techniques are limited by the observation that when cells are placed in artificial culture, they lose their specialized features, for example, as virulent bacterial cells capable of causing disease. Suspension cell culture is known to keep cells differentiated, that is, in their natural state, such as occurs in vivo. In suspension culture, cells are floated in liquid medium and rotated, or otherwise supported such that they are held in suspension without hitting the walls of their culture vessel. However, suspension cultures on the ground are subject to the force of gravity, causing the cells to fall out of suspension. In order to keep cells floating in liquid culture, gravity are offset by an equal and opposite force. Some culture systems do this by whirling the culture medium in which the cells are suspended for example, using a small propeller. However, this technique creates disruptive shear forces that can tear apart the cells, thereby causing them to lose their specialized features. Some of these problems are eliminated in suspension cultures carried out in a clinostat, such as the rotating wall vessel (RWV). Several lines of experimental evidence also suggest that in many cases, the true microgravity of space allows cells to regain their special features that can be lost in other means of suspension culture.

Methods of culturing cells or bacteria in microgravity are known to one of ordinary skill in the art. These include culture in normal gravity (such as on the Earth's surface, i.e. 1×g) in conditions which create simulated microgravity by balancing gravity with equal and opposite forces. One example of balancing gravity is applying forces in suspension culture optimized to minimize shear (such as in a RWV or other clinostat), which produces low-shear modeled microgravity (LSMMG). See for example U.S. Pat. Nos. 4,988,623; 5,026,650; and 5,153,131, which are incorporated by reference herein in their entirety. In the clinostat or RWV, shear is the predominant balancing force, with smaller components of other forces, such as centipedal force, Coriolus forces, and/or buoyancy. In other examples, gravity is offset by magnetic field, such as during magnetic levitation, or by buoyancy, such as with addition of Ficoll to a solution.

A clinostat, such as the RWV apparatus, is commercially available, such as from USA Synthecon (Houston, Tex.). When the axis of the RWV bioreactor's rotation is perpendicular to the gravitational vector, a condition of LSMMG is achieved. A condition of 1×g (normal gravity) is achieved in the RWV if the axis of rotation is parallel to the gravitational vector. In some examples, bacteria (such as S. enterica serovar Enteritidis) is cultured in simulated microgravity under LSMMG conditions. Control samples are cultured in normal gravity, such as in the RWV under 1×g conditions, or in standard suspension vessels.

Culture conditions for Salmonella are well known to one of ordinary skill in the art. Salmonella bacteria cultured for use in the described methods include, but are not limited to wild type S. enterica strain SL1344 (Gulig and Curtiss, Infect. Immun. 55:2891-2901, 1987) and isolates thereof, for example, strain λ339. See U.S. Pat. Nos. 4,888,170 and 6,706,472. Additional Salmonella bacteria strains are known to one of skill in the art, and include those available from the American Type Culture Collection (Manassas, Va.) and the Salmonella Genetic Stock Center (Calgary, Canada). Culture conditions include growth at 37° C. or ambient temperature (such as about 22° C. to about 27° C.) in Lennox broth or M9 medium. Salmonella may be cultured in volumes of about 1 ml to about 1000 ml, such as about 10 ml to about 50 ml culture volumes. Cells may be harvested after culture for time periods sufficient for differential RNA expression to occur, for example, growth for about 2 hours to about 96 hours, such as about 24 hours to about 72 hours, or about 48 hours. In a particular example, bacteria are harvested after growth for about 72 hours. Bacteria may also be harvested at defined growth stages, such as log phase, (a physiological state marked by back-to-back division cycles such that the population doubles in number every generation time; for example, mid log phase or mid-late log phase), or stationary phase (defined as a physiological point where the rate of cell division equals the rate of cell death, hence viable cell number remains constant). Conditions for culture of Salmonella in a clinostat or RWV system are known to one of skill in the art (see for example, Wilson et al., Proc. Natl. Acad. Sci. USA 99:13807-13812, 2002).

Although the clinostat (such as a RWV) is a good model system, in this system gravity is balanced rather than unloaded (as occurs in space), and this can limit the efficacy of microgravity simulations provided by this suspension culture device. To examine the effects of true microgravity, without these counterbalances (whirling or horizontally rotating), only the space environment can be used. Therefore, in additional examples, the methods described herein include culturing bacteria in microgravity where the microgravity is produced by spaceflight. This includes travel outside of the Earth's atmosphere, for example, in a space shuttle (such as a United States Space Shuttle or a Russian Soyuz vehicle), on the International Space Station, on a rocket or satellite, or other vehicle traveling outside the Earth's atmosphere. Spaceflight includes travel in Earth orbit, such as on the International Space Station or space shuttle.

Bacteria may be cultured in microgravity during spaceflight using hardware such as a fluid processing apparatus (FPA), which facilitates controlled growth conditions (such as addition of growth media, test compounds, or fixative) while maintaining suitable culture containment. An FPA consists of a glass barrel that contains a short bevel on one side and stoppers inside that separate individual chambers containing fluids used in the experiment. The glass barrel loaded with stoppers and fluids is housed inside a lexan sheath containing a plunger that pushes on the top stopper to facilitate mixing of fluids at the bevel. The bottom stopper in the glass barrel (and also the bottom of the lexan sheath) is designed to contain a gas-permeable membrane that allows air exchange during bacterial growth. In some examples, the bacteria cultured in microgravity produced by spaceflight are S. enterica serovar Enteritidis. The culture conditions include growth at 37° C. or at ambient temperature (such as about 22° C. to about 27° C.) in Lennox broth or M9 medium. Salmonella may be cultured in volumes of about 1 ml to about 100 ml, such as about 25 ml to about 50 ml culture volumes. In particular examples, Salmonella are cultured in a volume of about 3 ml. Cells may be harvested at particular time points, for example, growth for about 2 hours to about 96 hours, such as about 24 hours to about 72 hours, or about 48 hours. In a particular example, bacteria are harvested after growth for about 72 hours. In further examples, bacteria are cultured with C. elegans for virulence assays.

In additional examples, simulated microgravity may be achieved by offsetting gravity using a magnetic field, such as during magnetic levitation (see for example, Coleman et al., Biotechnol. Bioeng. 98:854-863, 2007; Dijkstra et al. In: Abstracts 11^{th} International Symposium on Microbial Ecology, Vienna, August 2006) or by offsetting gravity by buoyancy, such as addition of Ficoll to a solution (see for example, Coleman et al., Biotechnol. Bioeng. 2007 Dec. 13 (Epub ahead of print)).

C. RNAs

The methods of vaccine production provided herein include evaluating expression of RNAs in bacteria cultured in microgravity and detecting an RNA that is overexpressed or underexpressed during growth in microgravity as compared to growth in normal gravity, thereby creating a bacteria had has specific RNA deleted. By deleted it is meant that the bacteria no longer produce the functional RNA, for example due to a total, deletion, partial deletion, mutation that inhibits function, insertion that inhibits function, or any combination thereof. Bacteria are cultured substantially identically in microgravity and normal gravity conditions (such as substantially identical bacterial strains, culture times, temperatures, and growth media). Expression of RNAs is evaluated (such as with a microarray) in bacteria cultured in microgravity and bacteria cultured in normal gravity and expression levels of RNA are compared. The change in expression of an RNA may be expressed as the ratio of the amount of an RNA in bacteria cultured in microgravity to the amount of the same RNA in bacteria cultured in normal gravity. An increase in the ratio of the amount of RNA indicates an RNA that is overexpressed in bacteria cultured in microgravity as compared to bacteria cultured in normal gravity (such as a ratio of about 1.1 to about 100, for example about 1.5 to about 10). A decrease in the ratio of the amount of RNA indicates an RNA that is overexpressed in bacteria cultured in microgravity as compared to bacteria cultured in normal gravity (such as a ratio of about 0.90 to about 0.001, for example about 0.20 to about 0.60).

In some examples, the RNA which is differentially expressed during culture in microgravity is an RNA which is known to be associated with pathogenicity. In other examples, the differentially expressed RNA is an RNA which has not been previously associated with pathogenicity. The differentially expressed RNAs disclosed herein may be selected as having a particular characteristic produced by differential expression, for example an effect on bacterial virulence. In some examples, the differentially expressed RNA may increase or decrease bacterial virulence, either directly, or through its effects on other RNAs or proteins.

In some examples, the RNA which is overexpressed or underexpressed is a small RNA (sRNA). sRNAs are small molecular weight RNA that are typically encoded in the intergenic regions of bacteria chromosomes, for example in E. coli, S. enterica, or Y. pestis. sRNA are typically non-coding RNAs of about 50-500 nucleotides in length, which do not commonly contain an expressed open reading frame. It is estimated that enterobacterial genomes contain 200-300 sRNA genes (Vogel and Papenfort, Curr. Opin. Microbiol. 9:605-611, 2006). Methods for identifying sRNAs in bacteria are well known in the art (see for example Vogel and Sharma, Biol. Chem. 366:1219-1238, 2005).

Many sRNAs function by direct base-pairing with a target mRNA and affecting mRNA stability or ability to be translated. Most sRNAs are trans-encoded antisense RNAs (that is, they are encoded by a separate genetic locus that their target). However, some sRNAs are cis-encoded (that is, they are transcribed from the same locus as their target, but in the opposite orientation). The trans-encoded sRNAs often pair with their target mRNA by imperfect sequence complementarity. Some sRNAs act by inhibiting mRNA translation, such as by blocking ribosome entry (for example, MicA and SgrS act by this mechanism). Other sRNAs positively regulate mRNA translation, for example, by melting inhibitory secondary structures that sequester the ribosome entry site of mRNA (such as DsrA and RprA). In contrast, some sRNAs interact with proteins and modify their activity; for example, CsrB and CsrC sRNAs bind the translational regulatory protein CsrA and titrate it away from its mRNA target sites.

In some examples, the level of expression of sRNAs increases or decreases in bacteria cultured in microgravity as compared with bacteria cultured in normal gravity. Bacteria which overexpress a sRNA during culture in microgravity may exhibit increased virulence and provide suitable vaccine targets. In particular examples, the sRNA may include IstR, InvR, DsrA, SsrS, MicA, MicC, MicF, SroB, RybB, SraH, RprA, SgrS, GcvB, αRBS, rnaseP, csrB, tkel, oxyS, RFN, rne5 or any combination thereof. In some examples, the sRNA is a sRNA which inhibits mRNA translation, including but not limited to MicA, MicC, MicF, RybB, GcvB, SgrS, and DsrA. In other examples, the sRNA is a sRNA which increases mRNA translation, for example, RprA and DsrA.

In further examples, a sRNA-encoding nucleic acid which is overexpressed or underexpressed in bacteria cultured in microgravity is deleted from the bacteria. The resulting deleted bacterial strain is subsequently killed or attenuated to produce a vaccine. In particular examples, the deleted sRNA may include IstR, InvR, DsrA, SsrS, MicA, MicC, MicF, SroB, RybB, SraH, RprA, SgrS, GcvB, rnaseP, csrB, tkel, oxyS, RFN, rne5, or any combination thereof. In some examples, the deleted sRNA is a sRNA which inhibits mRNA translation, including but not limited to MicA, MicC, MicF, RybB, GcvB, SgrS, and DsrA. In other examples, the deleted sRNA is a sRNA which increases mRNA translation, such as RprA and DsrA.

In additional examples, the RNA which is overexpressed or underexpressed during culture in microgravity is an mRNA. In some examples, the change in expression level increases or decreases bacterial virulence. In particular examples, the mRNA is HilA, HilD, RhuM, PipA, or any combination thereof. HilA is a transcriptional activator of the Salmonella pathogenicity island-1 invasion genes. Exemplary nucleic acid sequences of HilA from Salmonella are available on GENBANK® at Accession Nos. NC_{—}003197, NC_{—}01129, and NC_{—}011274, herein incorporated by reference in their entirety as available Mar. 25, 2009. HilD is a transcriptional regulator that de-represses HilA. Exemplary nucleic acid sequences of HilD from Salmonella are available on GENBANK® at Accession Nos. NC_{—}003197, NC_{—}011294, and NC_{—}006511, herein incorporated by reference in their entirety as available Mar. 25, 2009. RhuM is a gene of unknown function located in Salmonella pathogenicity island-3. Exemplary nucleic acid sequences of RhuM from Salmonella are available on GENBANK® at Accession Nos. NC_{—}006511, NC_{—}011274 and NC_{—}003197, herein incorporated by reference in their entirety as available Mar. 25, 2009. PipA is a gene required for enteropathogenesis. Exemplary nucleic acid sequences of PipA from Salmonella are available on GENBANK® at Accession Nos. NC_{—}003198, NC_{—}011294, and NC_{—}011274, herein incorporated by reference in their entirety as available Mar. 25, 2009. See for example Tenor et al. Curr. Biol. 14:1018-1024, 2004. In further examples, a mRNA-encoding nucleic acid which is overexpressed or underexpressed in bacteria cultured in microgravity is deleted from the bacteria. In some examples, the resulting deleted bacterial strain is subsequently killed or attenuated to produce a vaccine. In particular examples, the deleted mRNA may include is HilA, HilD, RhuM, PipA, or any combination thereof.

In additional examples, the RNA that is overexpressed or underexpressed during culture in microgravity is an RNA encoding a gene given in Table 1. In some examples, a RNA given in Table 1 which is overexpressed or underexpressed in bacteria cultured in microgravity is deleted from the bacteria. In some examples, the resulting deleted bacterial strain is subsequently killed or attenuated to produce a vaccine. Thus, in some examples is at least one of the RNA encoding the genes listed in Table 1 is deleted from the bacteria, such as at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 2, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, or at least 90 of the RNAs encoding the genes listed in Table 1. In some examples, an RNA encoding an outer membrane protein is deleted from the bacteria, such as one or more of ompA, ompC, and ompD. In some examples, an RNA encoding part of the plasmid transfer apparatus is deleted from the bacteria, such as one or more of traB, traN, trbA, traK, traD, trbC, traH, traX, traT, trbB, traG, traF, and traR. In some examples, an RNA encoding a ribosomal protein is deleted from the bacteria, such as one or more of rpsL, rpsS, rplD, rpsF, rplP, rplA, rpme2 and rplY. In some examples, an iron utilization/storage associated RNA is deleted from the bacteria, such as one or more of adhE, entE, hydN, dmsC, nifU, fnr, fdnH, frdC, bfr, ompW and dps. In some examples, an RNA implicated in/associated with biofilm formation is deleted from the bacteria, such as one or more of wza, wcaI, ompA, wcaD, wcaH, manC, wcaG, wcaB, fimH, fliS, flgM, flhD, fliE, fliT, cheY and cheZ.

E. Detection of RNAs and Assessment of Bacterial Virulence

The methods described herein include evaluating the expression of RNA in bacteria cultured in microgravity and detecting an RNA that is overexpressed or underexpressed in a bacterial population during growth in microgravity as compared to growth in normal gravity. In some examples, the nucleic acid encoding the RNA is deleted to produce a deleted bacterial strain. In additional examples, the overexpression of an RNA results in an increase in the virulence of the bacteria cultured in microgravity.

i. Detecting RNA Expression

Methods for assessing expression levels of RNAs, such as mRNAs and sRNAs, are well known to one of ordinary skill in the art. For example, expression of RNAs may be assessed utilizing standard microarray techniques.

Microarrays which include probes from bacterial intergenic regions, where most sRNA genes reside, can be used to assess changes in sRNA expression in bacteria cultured in microgravity as compared with bacteria cultured in normal gravity. sRNAs can be profiled using microRNA microarrays (for example, miRCURY™ arrays, Exiqon, Inc., Woburn, Mass.). Microarrays which include both mRNAs and sRNAs may also be constructed by printing PCR amplicons representing about 99% of the genome of the desired bacteria on coated slides using an array maker (such as GeneMachine OmniGrid Array Maker, Genomic Instrumentation Services, San Carlos, Calif.).

Briefly, total RNA is prepared from bacterial samples that are cultured in microgravity (such as in a RWV in LSMMG mode, or during spaceflight) and in parallel conditions in normal gravity. The RNA is converted to cDNA and labeled (such as with a fluorescent dye). For example, cDNA generated from a sample cultured in microgravity may be labeled with Cy3, while cDNA generated from a sample cultured in normal gravity may be labeled with Cy5. Microarrays are probed by cohybridizing the differently labeled cDNA from microgravity and normal gravity samples with the array and scanned to detect the fluorescent signal (for example with GENECHIP® Scanner 3000, Affymetrix Inc.). The Cy3 and Cy5 values for each spot are normalized and the ratio of Cy3 to Cy5 fluorescence is determined. An increase in the ratio for a particular probe indicates that the RNA is overexpressed in bacteria that are cultured in microgravity, while a decrease in the ratio for a particular probe indicates that the RNA is underexpressed in bacteria that are cultured in microgravity.

Other methods of detecting RNA expression can also be used with the disclosed methods. For example, RNA expression and changes in RNA expression can be detected using PCR and/or Northern blots and the like.

ii. Methods for Assessing Virulence

In some examples, the overexpression or underexpression of an mRNA or sRNA during bacterial culture in microgravity conditions increases the virulence of the bacteria, for example increasing the virulence of the bacteria by at least 10%, such as at least 20%, at least 30%, at least 40%, at least 50%, at least 60%, at least 70%, at least 80%, at least 90%, at least 100%, at least 200%, at least 300%, at least 400%, or even greater than at least 500%. Methods for assessing virulence of a pathogen are well known in the art. In one example, assessing virulence includes determining microbial resistance to acid stress. For example, bacterial survival under conditions of acid stress (such as culture conditions of pH 3.5) can be determined. An increase in the percentage survival of bacteria in acid stress conditions indicates increased virulence, while a decrease in percent survival is an indicator of decreased virulence. An increase and conversely a decrease can be a change of at least 10%, such as at least 20%, at least 30%, at least 40%, at least 50%, at least 60%, at least 70%, at least 80%, at least 90%, at least 100%, at least 200%, at least 300%, at least 400%, or even greater than at least 500% in the percentage of bacteria.

Virulence may also be assessed by determining microbial resistance to killing following uptake by macrophages. In particular, the number of bacteria present inside macrophages (such as J774 macrophages) at time points following infection (such as 2 hours and 24 hours) is assessed. An increase in intracellular bacterial number in macrophages indicates an increase in virulence; a decrease in intracellular bacterial number in macrophages indicates decreased virulence.

Another means of determining microbial virulence is by assessing the killing of infected host organisms (such as mice or C. elegans). For example, virulence may be assessed by determining the lethal dose required to kill 50% of infected animals (LD₅₀), which is expressed in terms of the number of colony forming units (CFU) administered. In some examples, increased virulence includes a decrease in LD₅₀ (decreased CFU) and decreased virulence includes an increase in the LD₅₀ (increased CFU). Virulence may also be assessed in terms of the average time to death of an infected animal, such that a decrease in the average time to death indicates increased virulence, whereas an increase in the average time to death indicates decreased virulence. In some examples, the host organism may have gene deletions, such as C. elegans with deletion of MAPK/p38 or daf-2 genes.

Virulence may also be assayed by an invasion assay in C. elegans (see for example, Tenor and Aballay, EMBO Rep. 9:103-109, 2008). For example, C. elegans may be exposed to S. enterica which carries a green fluorescent protein (GFP) reporter gene (such as Smo22). Infected nematodes will exhibit the presence of GFP in the pharynx when examined by fluorescence microscopy. An increase in accumulation of GFP in the pharynx as compared to wild type Salmonella indicates increased virulence, while a decrease in accumulation of GFP in the pharynx indicates decreased virulence.

In additional examples, virulence may be assessed in cell culture models, such as bacterial invasion of or adhesion to cells in culture (including, Hep-2, Chinese hamster ovary, MDCK, and T84 cells). An increase in invasion or adhesion indicates increased virulence, while a decrease in invasion or adhesion indicates decreased virulence.

F. Creation of Bacterial Deletion Strains

The methods disclosed herein include deleting a nucleic acid encoding one or more RNAs that is differentially expressed in bacteria cultured in microgravity conditions as compared to bacteria cultured in normal gravity.

Methods for creating bacterial strains having deletions of nucleic acids are well known to one of ordinary skill in the art. One strategy is to replace a target bacterial nucleic acid of interest with another nucleic acid (such as one encoding a selectable antibiotic resistance gene, a green fluorescent protein encoding nucleic acid, or a transposon cassette). See U.S. Pat. Nos. 4,963,487 and 6,024,961); Datsenko and Wanner, Proc. Natl. Acad. Sci. USA 97:6640-6645, 2000. Methods for deleting more than one RNA in the same bacteria are also known in the art (Lambert et al., Appl. Environ. Microbiol. 73(4): 1126-35, 2007).

G. Vaccine Preparations and Modes of Administration

The vaccines produced by the methods disclosed herein include attenuated or killed vaccines. Methods for producing attenuated or killed vaccines are known to one of ordinary skill in the art. An attenuated vaccine, also referred to as “modified live,” refers to a living microorganism (for example, S. enterica), which has been attenuated (modified) by any of a number of methods known to one of ordinary skill in the art. These methods include, but are not limited to, multiple serial passage, temperature sensitive attenuation, mutation, or the like, such that the resultant strain is relatively non-pathogenic to a subject. The modified live strain should be capable of limited replication in the vaccinated subject and of inducing a protective immune response which is protective against disease caused by virulent or wild-type S. enterica.

A killed (or “inactivated”) vaccine is one in which the bacteria are treated by any of several means known to the art so that they no longer grow or reproduce, but are still capable of eliciting an immune response in the subject. Bacteria, such as S. enterica, may be killed using chemicals or enzymes, such as formalin, azide, detergent (for example, non-ionic detergents), phenol, thimerosal, propiolactone, lysozyme, or proteolytic enzymes. A killed or inactivated vaccine may also be produced by inactivating the bacteria by a physical treatment, such as heat treatment, freeze-thaw, sonication, or sudden pressure drop.

Methods of formulating and administering vaccine compositions are known to one of skill in the art. The attenuated or killed vaccines produced by the methods described herein are individually or jointly combined with a pharmaceutically acceptable carrier or vehicle for administration as an immunogen or vaccine to humans or animals. The immunogenic or vaccine formulations may be conveniently presented in bacterial colony forming units (CFU) unit dosage form and prepared using by conventional pharmaceutical techniques. Such techniques include the step of bringing into association the active ingredient and pharmaceutical carriers or excipient(s). In general, the formulations are prepared by uniformly and intimately bringing into association the active ingredient with liquid carriers. Formulations suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampoules and vials, and may be stored in a freeze-dried (lyophilized) condition requiring only the addition of the sterile liquid carrier, for example, water for injections, immediately prior to use. Extemporaneous injection solutions and suspensions may be prepared from sterile powders, granules and tablets commonly used by one of ordinary skill in the art.

Preferred unit dosage formulations are those containing a dose or unit, or an appropriate fraction thereof, of the administered ingredient. It should be understood that in addition to the ingredients particularly mentioned above, the formulations of the present invention may include other agents commonly used by one of ordinary skill in the art.

The immunogenic or vaccine composition may be administered through different routes, such as oral, including buccal and sublingual, rectal, parenteral, aerosol, nasal, intramuscular, subcutaneous, and intradermal, or a combination thereof. The composition may be administered in different forms, including but not limited to solutions, emulsions and suspensions, microspheres, particles, microparticles, nanoparticles, and liposomes. In some examples, administration of a vaccine may include administering a parenteral vaccine followed by oral dosing.

Administration can be accomplished by single or multiple doses. The dose administered to a subject in the context of the present disclosure should be sufficient to induce a beneficial therapeutic response in a subject over time, or to inhibit infection. The dose required will vary from subject to subject depending on the species, age, weight and general condition of the subject, the severity of the infection being treated, the particular vaccine being used (for example, a Salmonella bacterial strain having a deletion of a sRNA or mRNA), and its mode of administration. An appropriate dose can be determined by one of ordinary skill in the art using only routine experimentation.

It is expected that from about 1 to 5 dosages may be required per immunization regimen. Initial dosages may range from about 10⁴ to 10¹⁰ CFU, with a preferred range of about 10⁷ to 10¹⁰ CFU. Booster vaccination may be required and dosages may range from 10⁴ to 10⁹ CFU, with a preferred range of about 10⁶ to 10⁹ CFU. The volume of administration will vary depending on the route of administration. Intramuscular injections may range from about 0.1 ml to 1.0 ml.

The composition may be stored at temperatures of from about 100° C. to 4° C. The composition may also be stored in a lyophilized state at different temperatures, including room temperature. The composition may be sterilized through conventional means known to one of ordinary skill in the art. Such means include, but are not limited to filtration, radiation and heat. The composition may also be combined with bacteriostatic agents, such as thimerosal, to inhibit bacterial growth.

A variety of adjuvants known to one of ordinary skill in the art may be administered in conjunction with the vaccine composition. Such adjuvants include but are not limited to the following: polymers, co-polymers such as polyoxyethylene-polyoxypropylene copolymers, including block co-polymers; polymer P1005; Freund's complete adjuvant (for animals); Freund's incomplete adjuvant; sorbitan monooleate; squalene; CRL-8300 adjuvant; alum; QS 21, muramyl dipeptide; CpG oligonucleotide motifs and combinations of CpG oligonucleotide motifs; trehalose; bacterial extracts, including mycobacterial extracts; detoxified endotoxins; membrane lipids; or combinations thereof.

The present disclosure is illustrated by the following non-limiting Examples.

### EXAMPLES

### Example 1

### Culture of Salmonella

This example describes the culture conditions for growth of Salmonella in microgravity conditions in a RWV under LSMMG conditions and in spaceflight, and culture of control samples in normal gravity conditions.

The virulent, mouse-passaged S. typhimurium derivative of SL1344 termed λ339 was used as the WT strain in all flight- and ground-based trials. Isogenic derivatives of SL1344 with mutations Δhfq, hfq 3′Cm, and invA Km were used in ground-based trials. The Δhfq strain contains a deletion of the hfq ORF and replacement with a chloramphenicol resistance cassette, and the hfq 3′Cm strain contains an insertion of the same cassette immediately downstream of the WT hfq ORF. The invA Km strain contains a Km resistance cassette inserted in the invA ORF. Lennox broth was used as the growth medium in all cultures, and PBS was used to resuspend bacteria for use as inoculum in the FPAs. The RNA fixative RNA Later II (Ambion, Austin, Tex.), glutaraldehyde (16%; Sigma, St. Louis, Mo.), and formaldehyde (2%; Ted Pella, Redding, Calif.) were used as fixatives in flight trials.

Salmonella cultured in microgravity produced by spaceflight were grown in specially designed hardware, referred to as a fluid processing apparatus (FPA). Assembly of FPAs was carried out in normal gravity, prior to placement on the Space Shuttle. An FPA consists of a glass barrel that can be divided into compartments (by means of the insertion of rubber stoppers) and a lexan sheath into which the glass barrel is inserted. Each compartment in the glass barrel was filled with a solution in an order such that the solutions can be mixed at specific time points in flight via two actions: (i) downward plunging action on the rubber stoppers and (ii) passage of the fluid in a given compartment through a bevel on the side of the glass barrel such that it is released into the compartment below. Glass barrels and rubber stoppers are coated with a silicone lubricant (Sigmacote; Sigma) and autoclaved separately before assembly. A stopper with a gas exchange membrane was inserted just below the bevel in the glass barrel before autoclaving.

FPA assembly was performed aseptically in a laminar flow hood in the following order: 2.0 ml of Lennox broth medium on top of the gas-exchange stopper, one rubber stopper, 0.5 ml of PBS containing bacterial inoculum (about 6.7×10⁶ bacteria), another rubber stopper, 2.5 ml of either fixative (12% paraformaldehyde(final concentration 4%) or 6% glutaraldehyde (final concentration 2%)), RNA Later II (Ambion, Austin, Tex.), or Lennox broth medium, and a final rubber stopper. Syringe needles (gauge 25⅝) were inserted into rubber stoppers during this process to release air pressure and facilitate assembly. In some cases, the medium contains C. elegans and/or C. elegans eggs (N2 Bristol wild type, p38/MAPK deleted, daf-2 deleted, or TOL1 deleted strains).

The assembled FPAs were placed aboard the Space Shuttle and microgravity conditions are achieved by flight in Earth orbit. The cultures were initiated at day 2 of flight by depressing the FPA plunger to mix the bacterial inoculum with the Lennox broth medium (with or without C. elegans and/or C. elegans eggs). Cultures were grown at ambient temperatures (about 24-25° C.). At day 5 of flight, following incubation of the cultures for about 72 hours, the cultures were terminated by depressing the FPA plunger further to add RNA Later II, fixative, or additional Lennox broth medium. Parallel cultures, which contain the same bacterial strains and other components as those grown in space were grown contemporaneously in identical hardware under the same conditions in normal gravity on the Earth's surface as control samples. Control cultures were also grown under the same conditions in simulated microgravity using a clinostat (such as a RWV) which allows for use of identical FPA hardware.

Bacterial strains used include E. coli stain OP50 (as a food source), wild type S. enterica SL1344, and Smo22 (SL1344 expressing the green fluorescent protein cassette). Also included were four SL1344 strains each with a specific mRNA deletion (HilA, HilD, RhuM, or PipA) and a GFP cassette.

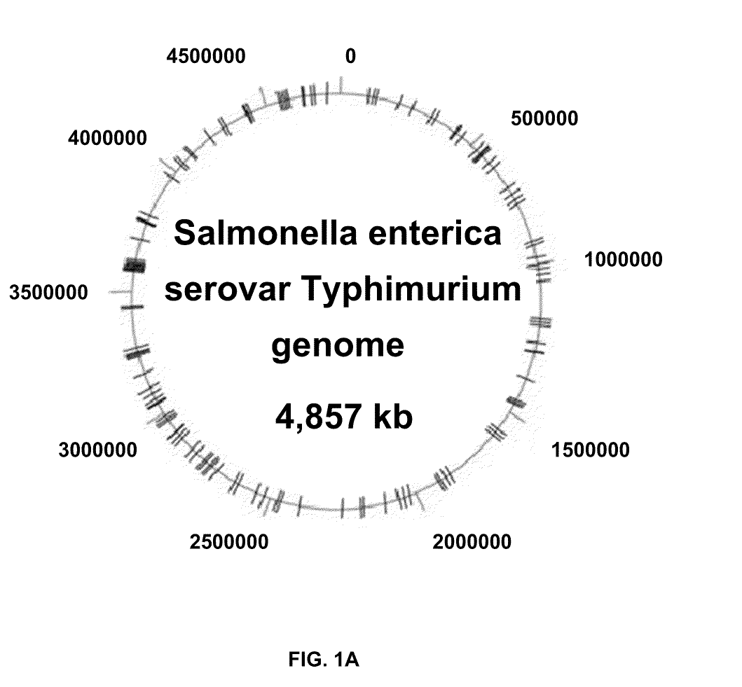

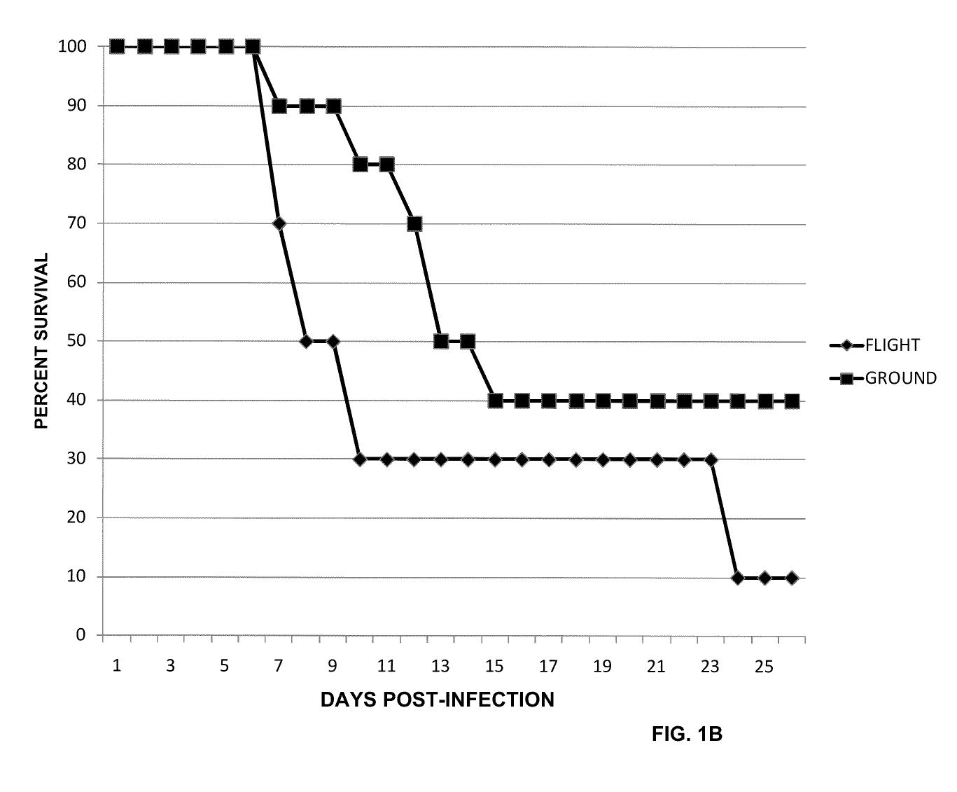

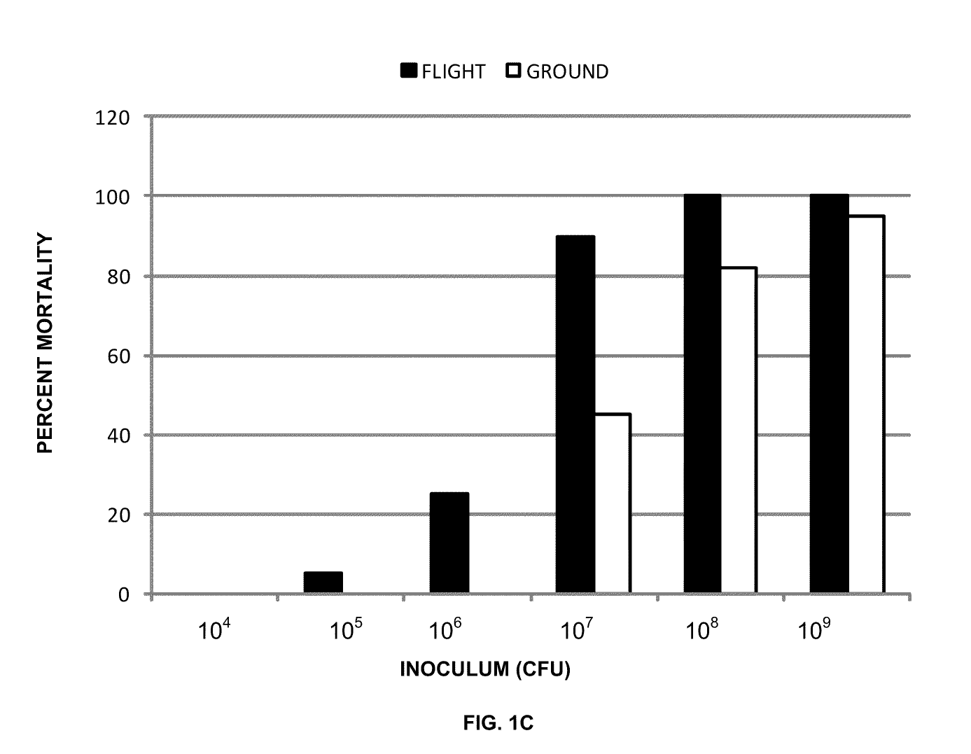

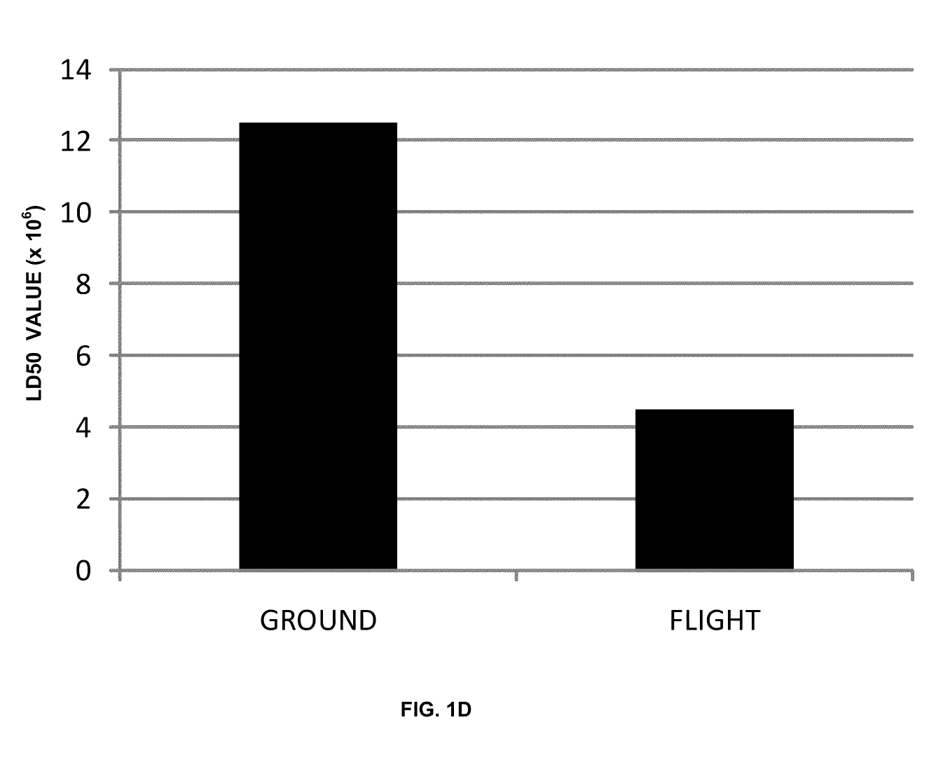

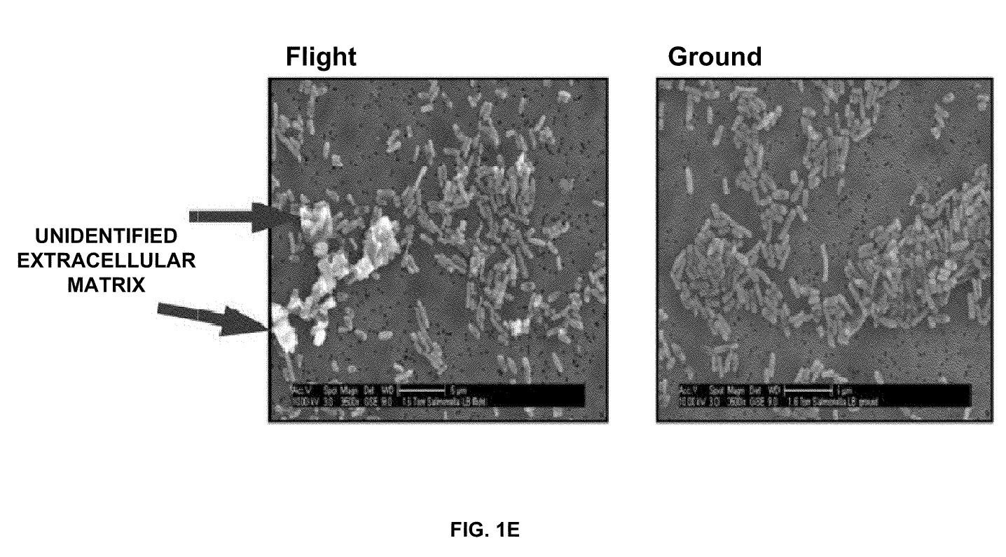

To determine any morphological differences between flight and ground cultures, SEM analysis of bacteria from these samples was performed. A portion of cells from the viable, media-supplemented cultures from flight and ground FPAs was fixed for SEM analysis (shown in FIG. 1E) by using 8% glutaraldehyde and 1% formaldehyde and was processed for SEM as described in Emami et al., Infect Immun 69:7106-7120, 2001. Although no difference in the size and shape of individual cells in both cultures was apparent, the flight samples demonstrated clear differences in cellular aggregation and clumping that was associated with the formation of an extracellular matrix (FIG. 1E). Consistent with this finding, several genes associated with surface alterations related to biofilm formation changed expression in flight (up-regulation of wca/wza colonic acid synthesis operon, ompA,fimH; down-regulation of motility genes) (see Table 1). SEM images of other bacterial biofilms show a similar matrix accumulation. Because extracellular matrix/biofilm formation can help to increase survival of bacteria under various conditions, this phenotype indicates a change in bacterial community potentially related to the increased virulence of the flight bacteria in the murine model.

### Example 2

### Microarray Analysis of RNA Expression

This example describes use of microarray analysis to detect sRNA or mRNAs that are overexpressed or underexpressed in Salmonella during culture in microgravity.

Samples which are cultured in microgravity and in normal gravity were used in microarray analysis of gene expression. Cultures were grown and terminated by mixing with RNALater II, as described in Example 1.

Total cellular RNA was obtained by using the QIAGEN® RNEASY® kit (QIAGEN®, Valencia, Calif.). Briefly, cells were harvested by centrifugation at 4° C., immediately resuspended in QIAGEN® RLT buffer, and lysed by agitation in the presence of glass beads. The RNA was then purified according to the manufacturer's instructions (QIAGEN®). Twenty micrograms of DNase-treated (AMBION®, Austin, Tex.) total RNA was converted to fluorescently labeled cDNA by using Fluorolink Cy3- or Cy5-dUTP (Amersham Pharmacia). To control for labeling differences, duplicate reactions were carried out where the Cy3 and Cy5 labels are switched during synthesis. Subsequent analysis of the two different labeling reactions was performed identically, as described below, by using the corresponding scan wavelength for each label during image acquisition.

sRNAs were analyzed with microarrays, such as the MIRCURY™ LNA Array v9.2 (Exiqon, Inc., Woburn, Mass.). Additional microarrays, which include both mRNA and sRNA, were prepared by printing PCR amplicons which represent approximately 99% of the S. enterica genome on aminosilane-coated slides by using a GeneMachine OMNIGRID® Array Maker (Genomic Instrumentation Services, San Carlos, Calif.). Each sample was printed in triplicate on each slide.

Immediately before use, the cDNA probes were resuspended in 50 μl of hybridization buffer (5×SSC/0.1% SDS/0.2 mg/ml BSA). Microarrays were probed by cohybridizing the fluorescently labeled microgravity and normal gravity cDNAs to the same microarray by using a GENOMIC SOLUTIONS® automated hybridization chamber (GENOMIC SOLUTIONS®, Ann Arbor, Mich.). Denatured probes were hybridized to slides for 18 hours (3 hours at 65° C., 3 hours at 55° C., and 12 hours at 50° C.). The slides were then washed twice with 2×SSC/0.1% SDS at 50° C., four times with 1×SSC at 42° C., and four times with 0.2×SSC at 42° C.

The microarrays were scanned for the Cy3 and Cy5 fluorescent signals by using SCANARRAY® 3000 from GSI Lumonics (General Scanning, Watertown, Mass.), and the stored images later analyzed by using IMAGENE® analysis software (Biodiscovery, Los Angeles) and GENESPRING™ software (Silicon Genetics, Palo Alto, Calif.). Data from stored array images were obtained with QUANTARRAY® software (Packard Bioscience, Billerica, Mass.) and statistically analyzed for significant gene expression differences by using the Webarray suite as described in Navarre et al., Science 313:236-238, 2006. GeneSpring software was also used to validate the genes identified with the Webarray suite.

To obtain the genes comprising the space flight stimulon, the following parameters were used in WEBARRAY™: a fold increase or decrease in expression of 2-fold or greater, a spot quality (A value) of >9.5, and P value of <0.05. For some genes listed in Table 1, the following parameters were used: a fold increase or decrease in expression of value>1.6 or <0.6, respectively, an A value of 8.5 or greater, and P value of <0.1. The vast majority of genes listed in Table 1 had an A value of >9.0 (with most being >9.5) and a P value of 0.05 or less. The exceptions were as follows: sbmA (P=0.06), oxyS (A=8.81), rplY (A=8.95), cspD (A=8.90), yfiA (P=0.08), ompX (P=0.09), hns (P=0.08), rmf (A=8.82), wcaD (P=0.09), and fliE (A=8.98). To identify space flight stimulon genes also contained in the Hfq regulon, proteins or genes found to be regulated by Hfq or RNAs found to be bound by Hfq as reported in the indicated references were scanned against the space flight microarray data for expression changes within the parameters above. Collectively, these gene expression changes form the first documented bacterial space flight stimulon indicating that bacteria respond to this environment with widespread alterations of expression of genes distributed globally throughout the chromosome (FIG. 1A).

TABLE 1
Space flight stimulon genes belonging to Hfq regulon or
involved with iron utilization or biofilm formation
Hfq regulon genes (up-regulated)
Outer membrane proteins

| | | |
|---|---|---|
| ompA | 2.05 | Outer membrane porin |
| ompC | 2.44 | Outer membrane porin |
| ompD | 3.34 | Outer membrane porin |

Plasmid transfer apparatus

| | | |
|---|---|---|
| traB | 4.71 | Conjugative transfer |
| traN | 4.24 | Conjugative transfer |
| trbA | 3.14 | Conjugative transfer |
| traK | 2.91 | Conjugative transfer |
| traD | 2.87 | Conjugative transfer |
| trbC | 2.68 | Conjugative transfer |
| traH | 2.59 | Conjugative transfer |
| traX | 2.37 | Conjugative transfer |
| traT | 2.34 | Conjugative transfer |
| trbB | 2.32 | Conjugative transfer |
| traG | 2.21 | Conjugative transfer |
| traF | 2.11 | Conjugative transfer |
| traR | 1.79 | Conjugative transfer |

Various cellular functions

| | | |
|---|---|---|
| gapA | 7.67 | Glyceraldehyde dehydrogenase |
| sipC | 6.27 | Cell invasion protein |
| adhE | 4.75 | Fe-dependent dehydrogenase |
| glpQ | 2.58 | Glycerophosphodiesterase |
| fliC | 2.11 | Flagellin, structural protein |
| sbmA | 1.67 | ABC superfamily transporter |

Hfq regulon genes (down-regulated)
Small RNAs

| | | |
|---|---|---|
| αRBS | 0.305 | Small RNA |
| rnaseP | 0.306 | Small RNA regulatory |
| csrB | 0.318 | Small RNA regulatory |
| tke1 | 0.427 | Small RNA |
| oxyS | 0.432 | Small RNA regulatory |
| RFN | 0.458 | Small RNA |
| rne5 | 0.499 | Small RNA |

Ribosomal proteins

| | | |
|---|---|---|
| rpsL | 0.251 | 30S ribosomal subunit protein S12 |
| rpsS | 0.289 | 30S ribosomal subunit protein S19 |
| rplD | 0.393 | 50S ribosomal subunit protein L4 |
| rpsF | 0.401 | 30S ribosomal subunit protein S6 |
| rplP | 0.422 | 50S ribosomal subunit protein L16 |
| rplA | 0.423 | 50S ribosomal subunit protein L1 |
| rpme2 | 0.473 | 50S ribosomal protein L31 |
| rplY | 0.551 | 50S ribosomal subunit protein L25 |

Various cellular functions

| | | |
|---|---|---|
| ynaF | 0.201 | Putative universal stress protein |
| ygfE | 0.248 | Putative cytoplasmic protein |
| dps | 0.273 | Stress response protein |
| hfq | 0.298 | Host factor for phage replication |
| osmY | 0.318 | Hyperosmotically inducible protein |
| mysB | 0.341 | Suppresses protein export mutants |
| rpoE | 0.403 | σE (σ24) factor |
| cspD | 0.421 | Similar to CspA; not cold-induced |
| nlpb | 0.435 | Lipoprotein-34 |
| ygaC | 0.451 | Putative cytoplasmic protein |
| ygaM | 0.453 | Putative inner membrane protein |
| gltI | 0.479 | ABC glutamate/aspartate transporter |
| ppiB | 0.482 | Peptidyl-prolyl isomerase B |
| atpE | 0.482 | Membrane-bound ATP synthase |
| yfiA | 0.482 | Ribosome-associated factor |
| trxA | 0.493 | Thioredoxin 1, redox factor |
| nifU | 0.496 | Fe—S cluster formation protein |
| rbfA | 0.506 | Ribosome-binding factor |
| rseB | 0.514 | Anti-σE factor |
| yiaG | 0.528 | Putative transcriptional regulator |
| ompX | 0.547 | Outer membrane protein |
| rnpA | 0.554 | RNase P, protein component |
| hns | 0.554 | DNA-binding protein |
| lamB | 0.566 | Phage λ receptor protein |
| rmf | 0.566 | Ribosome modulation factor |
| tpx | 0.566 | Thiol peroxidase |
| priB | 0.571 | Primosomal replication protein N |

Iron utilization/storage genes

| | | |
|---|---|---|
| adhE | 4.76 | Fe-dependent dehydrogenase |
| entE | 2.24 | 2,3-dihydroxybenzoate-AMP ligase |
| hydN | 2.03 | Electron transport (FeS center) |
| dmsC | 0.497 | Anaerobic DMSO reductase |
| nifU | 0.495 | Fe—S cluster formation protein |
| fnr | 0.494 | Transcriptional regulator, Fe-binding |
| fdnH | 0.458 | Fe—S formate dehydrogenase-N |
| frdC | 0.411 | Fumarate reductase, anaerobic |
| bfr | 0.404 | Bacterioferrin, iron storage |
| ompW | 0.276 | Outer membrane protein W |
| dps | 0.273 | Stress response protein and ferritin |

Genes implicated in/associated with biofilm formation

| | | |
|---|---|---|
| wza | 2.30 | Polysaccharide export protein |
| wcaI | 2.07 | Putative glycosyl transferase |
| ompA | 2.06 | Outer membrane protein |
| wcaD | 1.82 | Putative colanic acid polymerase |
| wcaH | 1.76 | GDP-mannose mannosyl hydrolase |
| manC | 1.71 | Mannose guanylyltransferase |
| wcaG | 1.68 | Bifunctional GDP fucose synthetase |
| wcaB | 1.64 | Putative acyl transferase |
| fimH | 1.61 | Fimbrial subunit |
| fliS | 0.339 | Flagellar biosynthesis |
| flgM | 0.343 | Flagellar biosynthesis |
| flhD | 0.356 | Flagellar biosynthesis |
| fliE | 0.438 | Flagellar biosynthesis |
| fliT | 0.444 | Flagellar biosynthesis |
| cheY | 0.461 | Chemotaxic response |
| cheZ | 0.535 | Chemotaxic response |

### Example 3

### Deletion of Selected RNAs

This example describes the production of Salmonella strains carrying a deletion of one or more sRNAs or mRNAs.

The Salmonella enterica SL1344 strain is used as wild-type strain. sRNA deletion derivates were constructed using the lambda-red recombinase method (Datsenko and Wanner, Proc Natl Acad Sci USA 97:6640-6645, 2000), and primer pairs specific for each sRNA, respectively. All chromosomal mutations are subsequently transferred to a fresh SL1344 background strain via P22 HT105/1 int-201 transduction (Schmieger, Mol Gen Genet. 110:378-381, 1971). In some examples, a kanamycin resistance cassette of plasmid pKD4 is used to disrupt the selected sRNAs. All gene deletions are verified by PCR with sRNA-specific primers. In some examples, mRNAs are disrupted by insertion of a TnphoA transposon cassette (Tenor et al. Curr. Biol. 14:1018-1024, 2004).

### Example 4

### Virulence of Bacteria Cultured under Microgravity Conditions

This example describes determination of changes in virulence of Salmonella cultured in microgravity using mice as a model system.

Virulence of Salmonella cultured under microgravity or normal gravity conditions was evaluated by determining the LD₅₀ in mice. Six- to eight-week-old female BALB/c mice were fasted for about 6 hours and then per-orally infected with increasing dosages of S. enterica harvested from flight and ground FPA cultures, which were resuspended in buffered saline gelatin. Ten mice per infectious dosage were used, and food and water were returned to the animals within 30 min after infection. The infected mice are monitored every 6-12 h for 30 days. The LD₅₀ value is calculated by using the formula of Reed and Muench (Am. J. Hyg. 27:493-497, 1938).

Because growth during space flight and potential global reprogramming of gene expression in response to this environment could alter the virulence of a pathogen, the virulence of S. typhimurium space flight samples was compared to identical ground controls. Bacteria from flight and ground cultures were harvested and immediately used to inoculate female BALB/c mice via a per-oral route of infection on the same day as the Shuttle landing. Mice infected with bacteria from the flight cultures displayed a decreased time to death (at the 107 dosage), increased percent mortality at each infection dosage, and a decreased LD₅₀ value compared with those infected with ground controls (FIG. 1B-1D). These data indicate increased virulence for space flight S. typhimurium samples and are consistent with previous studies in which the same strain of S. typhimurium grown in the RWV under LSMMG conditions displayed enhanced virulence in a murine model as compared with 1×g controls.

### Example 5

### Determining Vaccine Safety and Efficacy in an Animal Model

This example describes methods of determining the effectiveness of a candidate Salmonella vaccine using an animal model.

The safety and efficacy of Salmonella vaccines can be evaluated in animal models, such as mice, according to procedures well known in the art. By way of example, Salmonella vaccines are tested in adult and juvenile mice.

Sero-negative mice are injected intravenously or intramuscularly with various doses of Salmonella vaccine preparations, or vaccine preparations are administered orally or by intravenous or intraperitoneal injection. If the Salmonella vaccine preparations are being used as adjuvants, the mice may be pretreated with doses as previously described, as well as subcutaneously or intradermal. Mock-vaccinated animals serve as controls. The animals are monitored daily for clinical signs of illness, including weakness or any alteration of physical condition. At various time points post-inoculation, blood, serum or other body fluid samples can be taken to assay Salmonella-induced illness, anti-Salmonella antibody production, or other desired biological endpoints (for example, white blood cell count, red blood cell count, hematocrit, platelet count, or Salmonella content of spleen or blood). Moribund mice are euthanized and necropsied.

To test efficacy of the Salmonella vaccines, inoculated and sham-inoculated mice are administered wild-type Salmonella at various doses. Animals are observed daily for signs of clinical illness, weight loss and respiratory distress. Animals that are in distress or moribund are immediately anesthetized and then euthanized. As described above, at various time points following inoculation, small blood samples can be taken to test for the presence of Salmonella, such as Salmonella RNA. Serum samples can be collected to determine anti-Salmonella antibody titers.

### Example 6

### Safety and Efficacy of Salmonella Vaccines in Human Subjects

The safety and efficacy of Salmonella vaccines can be evaluated in human volunteers according to procedures well known in the art. Typically, human volunteers are selected from those having occupations that put them at risk of infection with Salmonella, such as poultry workers. All volunteers are screened to ensure they are in good health. Informed consent is obtained from each volunteer prior to vaccination.

In this example, human volunteers are injected with candidate Salmonella vaccine subcutaneously or intramuscularly at an appropriate dose. The appropriate dose is the dose approved by the FDA, and can be determined from suitable animal studies conducted prior to human vaccination trials. Other routes of administration are possible, including intramuscular and intravenous. The vaccine can be administered as a single dose, or given in multiple doses, such as two, three or four doses. When administered in multiple doses, the booster doses can be administered at various time intervals, such as months to years. Serum samples can be obtained to determine antibody titers and identify responder and non-responders to the vaccine.

Vaccinated volunteers are encouraged to return and report local or systemic reactions. Local reactions are assessed by grading pain and tenderness at the site of inoculation and/or axillary lymph nodes and measuring erythema and induration at the site. Systemic reaction parameters include fever, chills, headache, malaise, myalgia, arthralgia, sore throat, gastric upset, dizziness, photophobia and skin rash. Additional laboratory samples, including complete blood cell count, chemistry profile, urinalysis, and blood samples for bacterial titrations can be obtained. Vaccinated volunteers are also screened for the development of Salmonella infection.

In view of the many possible embodiments to which the principles of the disclosure may be applied, it should be recognized that the illustrated embodiments are only examples of the disclosure and should not be taken as limiting the scope of the invention. Rather, the scope of the invention is defined by the following claims. We therefore claim as our invention all that comes within the scope and spirit of these claims.

We claim:

1. A method of identifying immunogenic compositions, the method comprising:
culturing bacteria in microgravity;
evaluating expression of RNA in the bacteria cultured in microgravity;
detecting an RNA that is differentially expressed in the bacteria during growth in microgravity as compared to growth in normal gravity;
selecting the bacteria cultured in microgravity that differentially express the RNA; and
determining virulence of the selected bacteria, wherein altered virulence of the selected bacteria as compared to bacteria cultured in normal gravity identifies an immunogenic composition.

2. The method of claim 2, wherein the differentially expressed RNA alters virulence.

3. The method of claim 1, wherein the bacteria is a mammalian pathogen.

4. The method of claim 3, wherein the mammalian pathogen is a species of comprises Enterobacter, Escherichia, Klebsiella, Proteus, Salmonella, Shigella, Yersinia, Staphylococcus, Streptococcus, Enterococcus, or Pseudomonas.

5. The method of claim 3, wherein the bacteria comprises bacteria from family Enterobacteriaceae.

6. The method of claim 5, wherein the bacteria comprises Salmonella enterica serovar Enteritidis.

7. The method of claim 1, wherein the RNA comprises a small RNA.

8. The method of claim 7, wherein the small RNA comprises IstR, InvR, DsrA, SsrS, MicA, MicC, MicF, SroB, RybB, SraH, RprA, SgrS, GcvB, or any combination thereof.

9. The method of claim 1, wherein the RNA comprises a messenger RNA.

10. The method of claim 9, wherein the messenger RNA comprises HilA, HilD, RhuM, PipA, or any combination thereof.

11. The method of claim 1, wherein the differential expression of RNA comprises overexpression.

12. The method of claim 1, wherein the differential expression of RNA comprises underexpression.

13. The method of claim 1, wherein the evaluating expression of the RNA comprises microarray analysis.

14. The method of claim 1, wherein the microgravity is produced by spaceflight.

15. A method for producing an immunogenic composition, the method comprising:
culturing bacteria in microgravity;
evaluating expression of RNA in the bacteria cultured in microgravity;
detecting an RNA that is differentially expressed in the bacteria during growth in microgravity as compared to growth in normal gravity;
deleting a nucleic acid encoding the detected RNA that is differentially expressed in the bacteria, thereby producing a deleted bacterial strain; and
attenuating or killing the deleted bacterial strain, thereby producing the immunogenic composition.

16. The method of claim 15, wherein the differentially expressed RNA alters virulence.

17. The method of claim 16, wherein the bacteria is a mammalian pathogen.

18. The method of claim 17, wherein the mammalian pathogen a species of Enterobacter, Escherichia, Klebsiella, Proteus, Salmonella, Shigella, Yersinia, Staphylococcus, Streptococcus, Enterococcus, or Pseudomonas.

19. The method of claim 15, wherein the bacteria comprises bacteria from family Enterobacteriaceae.

20. The method of claim 19, wherein the bacteria comprises Salmonella enterica serovar Enteritidis.

21. The method of claim 15, wherein the RNA comprises a small RNA.

22. The method of claim 21, wherein the small RNA comprises IstR, InvR, DsrA, SsrS, MicA, MicC, MicF, SroB, RybB, SraH, RprA, SgrS, GcvB, or any combination thereof.

23. The method of claim 15, wherein the RNA comprises a messenger RNA.

24. The method of claim 23, wherein the messenger RNA comprises HilA, HilD, RhuM, PipA, or any combination thereof.

25. The method of claim 15, wherein the differential expression of RNA comprises overexpression.

26. The method of claim 15, wherein the differential expression of RNA comprises underexpression.

27. The method of claim 15, wherein the evaluating expression of the RNA comprises microarray analysis.

28. The method of claim 15, wherein the microgravity is produced by spaceflight.

29. An immunogenic composition produced by the method of claim 15.

30. The immunogenic composition of claim 29, further comprising a pharmaceutically acceptable carrier.

31. The immunogenic composition of claim 30, further comprising an adjuvant.

32. An immunogenic composition comprising Salmonella enterica deleted for a nucleic acid encoding a RNA, wherein the RNA is selected from the group consisting of IstR, InvR, DsrA, SsrS, MicA, MicC, MicF, SroB, RybB, SraH, RprA, SgrS, GcvB, HilA, HilD, RhuM, PipA, or any combination thereof.

33. A method for producing an immunogenic composition, the method comprising:
culturing Salmonella enterica in microgravity conditions;
evaluating expression of RNA in the bacteria cultured in microgravity;
detecting an RNA that is differentially expressed in the bacteria during growth in microgravity as compared to growth in normal gravity, wherein the differential expression alters virulence of the S. enterica;
deleting a nucleic acid encoding the detected RNA that is differentially expressed in the S. enterica, thereby producing a deleted strain; and
attenuating or killing the deleted strain, thereby producing the immunogenic composition.
